(19) United States
(12) Patent Application Publication
(10) Pub. No.: US 2025/0213800 A1
(43) Pub. Date: Jul. 3, 2025

(54) INHALATION DEVICE

(71) Applicant: TRANSPIRE BIO INC., Weston, FL (US)

(72) Inventors: Pan XIE, Shenzhen (CN); Shiyi CHENG, Shenzhen (CN); Jun LI, Shenzhen (CN)

(21) Appl. No.: 18/987,614

(22) Filed: Dec. 19, 2024

(30) Foreign Application Priority Data
Dec. 29, 2023 (CN) 202323666157.1

(51) Int. Cl. A61M 11/00

(52) U.S. Cl. CPC A61M 11/003

(57) ABSTRACT

This application provides an inhalation device, and the device includes: a housing; an atomization structure, arranged in the housing and including a mist outlet; a cover body, movably connected to the housing; and a blocking member, arranged on the cover body, where when the cover body is configured to be in a first state, the blocking member is attached to the mist outlet and blocks the mist outlet; and when the cover body is configured to be in a second state, the blocking member is separated from the mist outlet. In this way, when the inhalation device is in use, the cover body is configured to be in the second state, and when the inhalation device is not in use, the cover body is configured to be in the first state. When the cover body is configured to be in the first state, the blocking member is attached to the mist outlet and blocks the mist outlet, so that an atomization medium in the housing is not evaporated through the mist outlet after passing through the atomization structure, thereby reducing a problem of a large deviation in an amount of mist sprayed.

### TECHNICAL FIELD

The present disclosure relates to the field of atomization technologies, and in particular, to an inhalation device.

### BACKGROUND

In the field of atomization technologies, an atomization material is generally atomized into an aerosol by using an inhalation device for a user to inhale. In a conventional inhalation device, the atomization material in a liquid storage bottle is inhaled into an atomization structure, and an aerosol for inhalation by the user is formed through atomization by the atomization structure.

However, the amount of mist sprayed out by an existing inhalation device each time is inconsistent. Particularly, when the inhalation device is used again after being unused for a period of time, there may be a large deviation in the amount of mist sprayed out, and the inhalation device needs to spray out mist for a plurality of times in advance before the inhalation device can spray out a normal mist amount.

### SUMMARY

Technical problems mainly resolved by this application are that the amount of mist sprayed out by an inhalation device each time is inconsistent, and an atomization material may evaporate.

To resolve the foregoing technical problems, a technical solution adopted in this application is as follows: an inhalation device is provided. The inhalation device includes: a housing; an atomization structure, arranged in the housing and including a mist outlet; a cover body, movably connected to the housing; and a blocking member, arranged on the cover body, where when the cover body is configured to be in a first state, the blocking member is attached to the mist outlet and blocks the mist outlet; and when the cover body is configured to be in a second state, the blocking member is separated from the mist outlet.

In an embodiment, the blocking member is detachably connected to the cover body.

In an embodiment, a first receiving groove is provided on the inner wall surface of the cover body, and the blocking member is embedded in the first receiving groove; or a second receiving groove is provided on the blocking member, a convex clamping block is arranged on the inner wall surface of the cover body, and the clamping block is embedded in the second receiving groove.

In an embodiment, a first magnetic member is arranged on the cover body, a second magnetic member is arranged on the blocking member, and the blocking member is magnetically connected to the cover body.

In an embodiment, the surface of the blocking member includes a bump, where when the cover body is configured to be in the first state, the bump is embedded in the mist outlet, and is in an interference fit with the mist outlet.

In an embodiment, the atomization structure includes: a mounting base, including a mounting cavity, where the mounting cavity includes a liquid inlet and the mist outlet; at least two microfluidic bodies, arranged in the mounting cavity; and a sealing member, arranged in the mounting cavity and sealing the at least two microfluidic bodies, where a liquid inlet end of the microfluidic body is in communication with the liquid inlet, and a mist outlet end of the microfluidic body is in communication with the mist outlet, to cause an aerosol-generating material entering from the liquid inlet to be sprayed out from the mist outlet after being atomized by the at least two microfluidic bodies.

In an embodiment, the sealing member includes at least two receiving holes, and the at least two microfluidic bodies are arranged in the at least two receiving holes in a one-to-one correspondence; the mounting cavity includes a top wall and a bottom wall, where the top wall of the mounting cavity includes the mist outlet, and the bottom wall of the mounting cavity includes the liquid inlet; and the sealing member and the bottom wall of the mounting cavity are spaced apart and cooperate to form a shunting cavity; and the two receiving holes are respectively located on two opposite sides of an extension line of the liquid inlet, and are separately in communication with the liquid inlet through the shunting cavity.

In an embodiment, the mounting base includes: a first mounting base, including a mounting groove; and a second mounting base, covering the mounting groove to form the mounting cavity, where a flange is arranged on the surface of the second mounting base close to the first mounting base, the flange is inserted into the mounting groove, and the sealing member abuts against the flange and is spaced apart from the surface of the second mounting base close to the first mounting base.

In an embodiment, the atomization structure further includes: a holder, including a receiving cavity, where the mounting base is arranged in the receiving cavity; and the receiving cavity includes a liquid inlet channel in communication with the liquid inlet, and an avoidance opening for exposing the mist outlet; and a filter element, arranged at a joint between the liquid inlet channel and the liquid inlet, where the liquid inlet channel is in communication with the liquid inlet through the filter element.

In an embodiment, the housing includes a mist outlet channel, one end of the atomization structure is located in the mist outlet channel and includes the mist outlet, and the mist outlet is spaced apart from a port of the mist outlet channel; and the cover body is rotatably connected to the housing, where when the cover body is configured to be in the first state, the cover body covers the mist outlet channel and is engaged with the housing; and when the cover body is configured to be in the second state, the mist outlet channel is exposed.

Beneficial effects of this application are as follows: different from the related art, this application provides an inhalation device, and the device includes: a housing; an atomization structure, arranged in the housing and including a mist outlet; a cover body, movably connected to the housing; and a blocking member, arranged on the cover body, where when the cover body is configured to be in a first state, the blocking member is attached to the mist outlet and blocks the mist outlet; and when the cover body is configured to be in a second state, the blocking member is separated from the mist outlet. In this way, when the inhalation device is in use, the cover body is configured to be in the second state, and when the inhalation device is not in use, the cover body is configured to be in the first state. When the cover body is configured to be in the first state, the blocking member is attached to the mist outlet and blocks the mist outlet, so that an atomization material in the housing does not evaporate through the mist outlet after passing through the atomization structure, thereby reducing a problem of a large deviation in the amount of mist sprayed out.

### BRIEF DESCRIPTION OF THE DRAWINGS

FIG. 1 is a schematic diagram of an overall structure of an inhalation device according to an embodiment of this application;

FIG. 2 is a cross-sectional view of an inhalation device in an A-A direction according to an embodiment of this application;

FIG. 3 is a schematic diagram of a cover body, a blocking member, and a housing after being separated according to an embodiment of this application;

FIG. 4a is an overall schematic diagram of a cover body from a first perspective according to an embodiment of this application;

FIG. 4b is a side view of a blocking member according to an embodiment of this application;

FIG. 5a is an enlarged view at Q in FIG. 2;

FIG. 5b is an enlarged view of a cover body at Q in FIG. 2 after being lifted;

FIG. 6 is a cross-sectional view of an atomization structure in an A-A direction according to an embodiment of this application;

FIG. 7 is an enlarged view at P in FIG. 6;

FIG. 8 is an overall schematic diagram of a sealing member from a first perspective according to an embodiment of this application;

FIG. 9 is an overall schematic diagram of a sealing member from a second perspective according to an embodiment of this application;

FIG. 10 is an overall schematic diagram of a first mounting base according to an embodiment of this application;

FIG. 11 is an overall schematic diagram of a second mounting base from a first perspective according to an embodiment of this application; and

FIG. 12 is an overall schematic diagram of a second mounting base from a second perspective according to an embodiment of this application.

List of Reference Numerals: 1—Inhalation device; 2—Atomization structure; 21—Sealing member; 211—Receiving hole; 212—First shunting groove; 22—Mounting base; 221—Mounting cavity; 2211—Liquid inlet; 2212—Mist outlet; 2213—Shunting cavity; 2213a—Sub-shunting cavity; 222—First mounting base; 2221—Mounting groove; 2222—Limiting groove; 223—Second mounting base; 2231—Flange; 2232—Convex ring; 2232a—Common side wall; 2232b—Opening; 2233—Limiting side wall; 23—Microfluidic body; 231—Liquid inlet end; 232—Mist outlet end; 3—Housing; 31—Mist outlet channel; 311—Port; 4—Liquid guide tube; 5—Cover body; 51—First receiving groove; 6—Blocking member; 61—Bump; 7—Liquid storage bottle; 8—Holder; 81—Receiving cavity; 82—Liquid inlet channel; 83—Avoidance opening; 84—Groove; and 85—Filter element.

### DETAILED DESCRIPTION

The technical solutions in the embodiments of this application are clearly and completely described below with reference to the accompanying drawings in the embodiments of this application. The described embodiments are merely some rather than all of the embodiments of this application. Based on the embodiments of this application, all other embodiments obtained by a person of ordinary skill in the art without creative efforts shall fall within the protection scope of this application.

The terms “first”, “second”, and “third” in this application are merely intended for a purpose of description and shall not be understood as indicating or implying relative significance or implicitly indicating the number of indicated technical features. Therefore, features defined with “first”, “second”, and “third” can explicitly or implicitly include at least one of the features. In the description of this application, “a plurality of” means at least two, such as two and three unless it is specifically defined otherwise. All directional indications (for example, up, down, left, right, front, back) in the embodiments of this application are only used for explaining relative position relationships, movement situations, or the like between the various components in a specific posture (as shown in the accompanying drawings). If the specific posture changes, the directional indications change accordingly. In addition, terms “comprise”, “include”, and any variations thereof are intended to indicate non-exclusive inclusion. For example, a process, method, system, product, or device that includes a series of steps or units is not limited to the listed steps or units; and instead, further optionally includes a step or unit that is not listed, or further optionally includes another step or unit that is intrinsic to the process, method, product, or device.

“Embodiment” mentioned in this specification means that particular features, structures, or characteristics described with reference to the embodiment may be included in at least one embodiment of this application. The term appearing at different positions of the specification may not refer to the same embodiment or an independent or alternative embodiment that is mutually exclusive with another embodiment. A person skilled in the art explicitly or implicitly understands that the embodiments described in this specification may be combined with other embodiments.

In the related art, the amount of mist sprayed out by an inhalation device may be inconsistent each time. Particularly, after the inhalation device is unused for a long period of time, the inhalation device may even fail to spray out vapor. The inventor of this application found that a spray port of an existing inhalation device is not sealed. As a result, after an appliance is started for use, an atomization material evaporates from the spray port to different extents due to different environmental factors, resulting in inconsistency in the amount of mist sprayed out each time. After being unused for a long period of time, precipitation of the solute in a medical liquid may further block a microfluidic channel in a microfluidic body, resulting in a reduced amount of mist sprayed out, or even a case in which mist cannot be sprayed out.

Based on this, an embodiment of this application provides an inhalation device, which effectively improves a problem of inconsistent amount of mist sprayed out each time and evaporation of the atomization material.

This application is further described in detail below with reference to the accompanying drawings and embodiments.

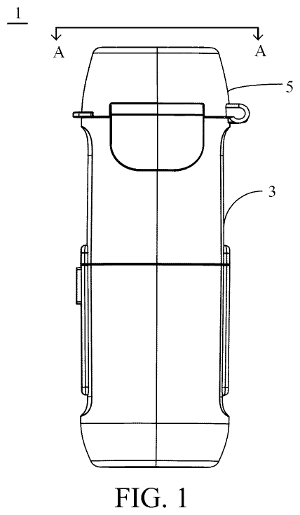

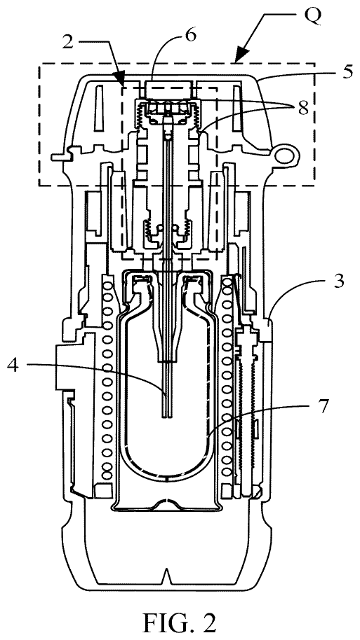

Referring to FIG. 1 and FIG. 2, FIG. 1 is a schematic diagram of an overall structure of an inhalation device according to an embodiment of this application; and FIG. 2 is a cross-sectional view of an inhalation device in an A-A direction according to an embodiment of this application.

An embodiment of this application provides an inhalation device 1. The inhalation device 1 includes an atomization structure 2, a housing 3, and a liquid guide tube 4. The atomization structure 2 and the liquid guide tube 4 are arranged in the housing 3, one end of the liquid guide tube 4 is in communication with the atomization structure 2, and the other end of the liquid guide tube 4 is configured to communicate with a liquid storage bottle 7. When a user uses the inhalation device 1, an atomization material in the liquid storage bottle 7 flows to the atomization structure 2 through the liquid guide tube 4, and then is atomized by the atomization structure 2 to form an aerosol for the user to inhale. The inhalation device 1 may be used in the fields such as medical atomization, recreation inhalation, and cosmetic atomization. The inhalation device 1 may include a liquid material stored in the liquid storage bottle 7, for example, an oil or a medical liquid to which an effective component is added.

Specifically, the inhalation device 1 further includes a cover body 5 and a blocking member 6.

The atomization structure 2 includes a mist outlet 2212, and the atomization material is atomized by the atomization structure 2 and then sprayed out from the mist outlet 2212 for the user to inhale.

The cover body 5 is movably connected to the housing 3, for example, may be rotatably connected, slidably connected, or pluggably connected to the housing 3. The cover body 5 is configured to protect a mist outlet channel 31, the mist outlet 2212, and the like, to prevent damage after the mist outlet channel 31 and the mist outlet 2212 are scraped with another device. In addition, after the cover body 5 covers the housing 3, it is convenient for the user to carry the inhalation device 1.

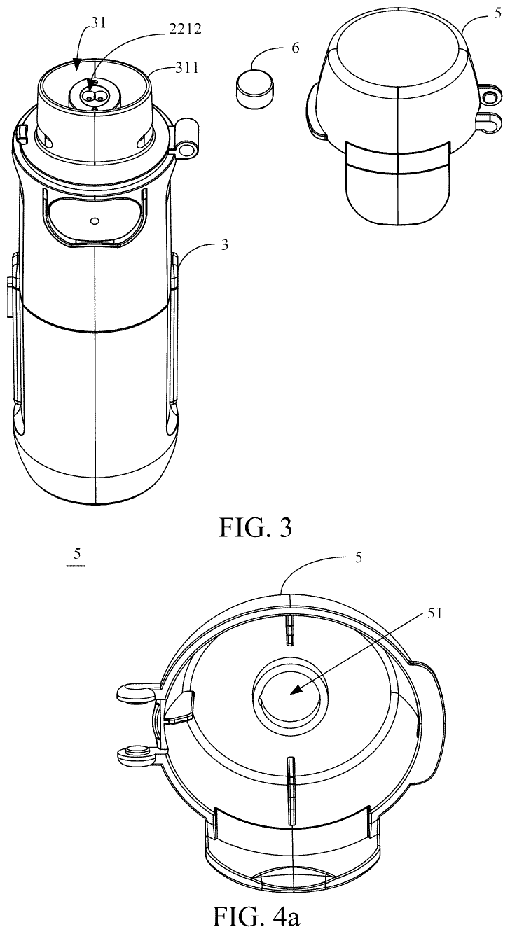

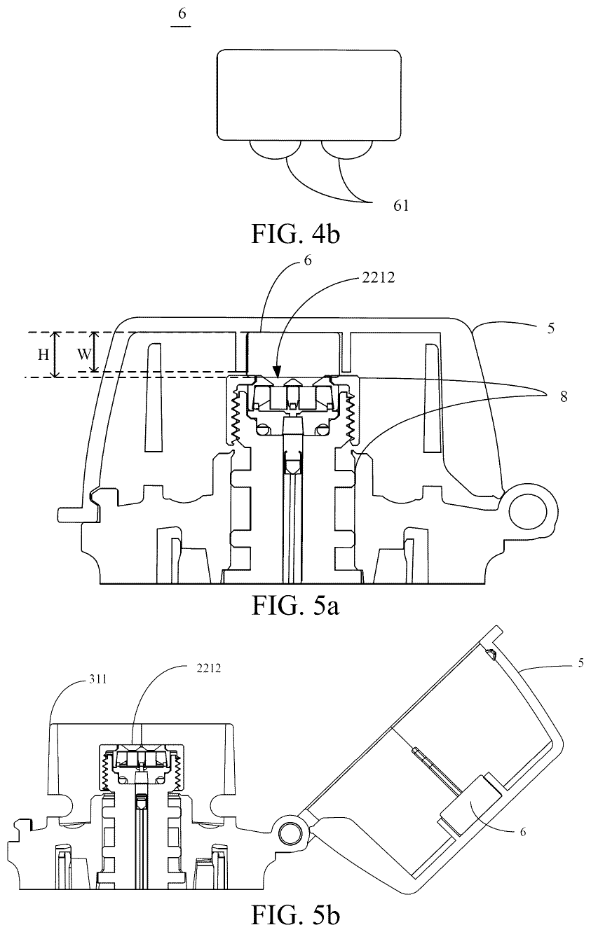

As shown in FIG. 2 to FIG. 4a, FIG. 3 is a schematic diagram of a cover body, a blocking member, and a housing after being separated according to an embodiment of this application, and FIG. 4a is an overall schematic diagram of a cover body from a first perspective according to an embodiment of this application. The blocking member 6 is arranged on the cover body 5 and is movable with the cover body 5. The blocking member 6 may be in a shape of a cylinder, a cube, a cuboid, or the like. The area of the end surface of the blocking member 6 close to the mist outlet 2212 is greater than or equal to the area of the mist outlet 2212. As shown in FIG. 5a and FIG. 5b, FIG. 5a is an enlarged view at Q in FIG. 2, and FIG. 5b is an enlarged view of the cover body at Q in FIG. 2 after being lifted. When the cover body 5 is configured to be in a first state, the blocking member 6 is attached to the mist outlet 2212 of the atomization structure 2 and blocks the mist outlet 2212. As shown in FIG. 5b, when the cover body 5 is configured to be in a second state, the blocking member 6 is separated from the mist outlet 2212. The blocking member 6 may be made of a flexible material such as silicone.

In an embodiment, the blocking member 6 is detachably connected to the cover body 5. In this way, after the blocking member 6 is detached from the cover body 5, replacement of a new blocking member 6 is facilitated, and stains on the blocking member 6 can be cleaned.

In an embodiment, as shown in FIG. 4a, a first receiving groove 51 is provided on the inner wall surface of the cover body 5, and the blocking member 6 is embedded in the first receiving groove 51. The first receiving groove 51 and the blocking member 6 are fixed by screws, an interference fit, or bonding. Alternatively, the circumferential length of the inner wall surface of the first receiving groove 51 is slightly less than the circumferential length of the blocking member 6, so that a slotted hole of the first receiving groove 51 squeezes the blocking member 6 in a circumferential direction, in other words, the first receiving groove 51 is in an interference fit with the blocking member 6. Alternatively, a second receiving groove (not shown in the figure) is provided on the blocking member 6, a convex clamping block (not shown in the figure) is arranged on the inner wall surface of the cover body 5, and the clamping block is embedded in the second receiving groove. Similarly, the clamping block of the cover body 5 is in an interference fit with the second receiving groove. In this way, the blocking member 6 may be movable with the cover body 5.

As shown in FIG. 5a, the length H of the blocking member 6 in an axial direction is larger than the depth W of the first receiving groove 51, and the end surface of the blocking member 6 away from the cover body 5 protrudes from the first receiving groove 51. In this way, when the cover body 5 is configured to be in the first state, the cover body 5 abuts against the mist outlet 2212 and blocks the mist outlet 2212. In an embodiment, a first magnetic member (not shown in the figure) is arranged on the cover body 5, a second magnetic member (not shown in the figure) is arranged on the blocking member 6, and the blocking member 6 is magnetically connected to the cover body 5. In this way, the blocking member 6 may be movable with the cover body 5, and the cover body 5 can be easily disassembled for replacement and cleaning.

In an embodiment, as shown in FIG. 4b, FIG. 4b is a side view of a blocking member according to an embodiment of this application. The surface of the blocking member 6 includes a bump 61. When the cover body 5 is configured to be in the first state, the bump 61 is embedded in the mist outlet 2212, and is in an interference fit with the mist outlet 2212. In this way, when the cover body 5 is configured to be in the first state, the blocking member 6 blocks the mist outlet 2212, and a blocking effect is better.

As described above, the blocking member 6 is arranged on the cover body 5. When the inhalation device 1 is in use, the cover body 5 is configured to be in the second state, and the blocking member 6 is separated from the mist outlet 2212. After the inhalation device 1 is not in use, the cover body 5 is configured to be in the first state. In this way, when the cover body 5 is configured to be in the first state, the blocking member 6 is attached to the mist outlet 2212 and blocks the mist outlet 2212, so that the atomization material in the housing 3 does not evaporate through the mist outlet 2212 after passing through the atomization structure 2. In addition, the mist outlet 2212 can be prevented from being polluted, and a problem of a large deviation in the amount of mist sprayed out can be reduced, to maintain consistency in an amount of atomization as much as possible. In addition, when the inhalation device 1 is used again after being unused for a period of time, the deviation in the amount of mist sprayed out is small, and there are fewer pre-sprays, thereby improving user experience.

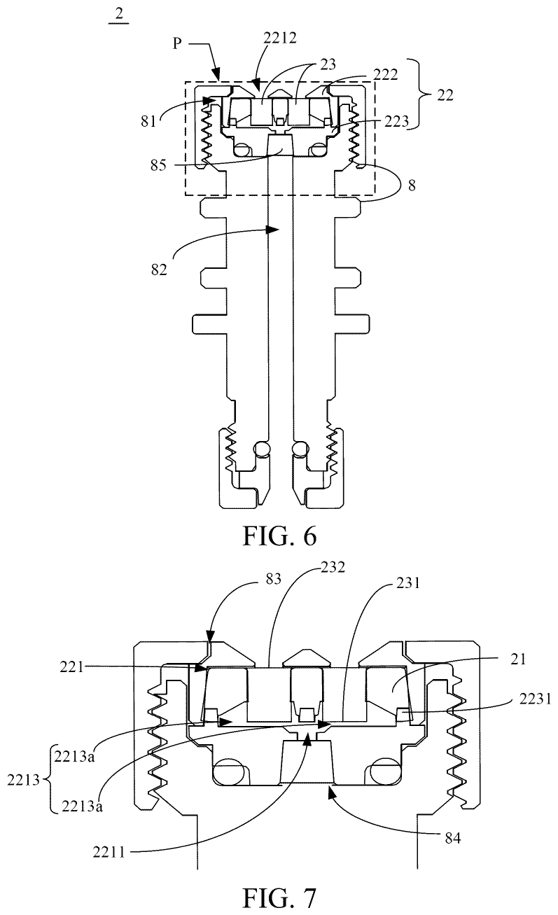

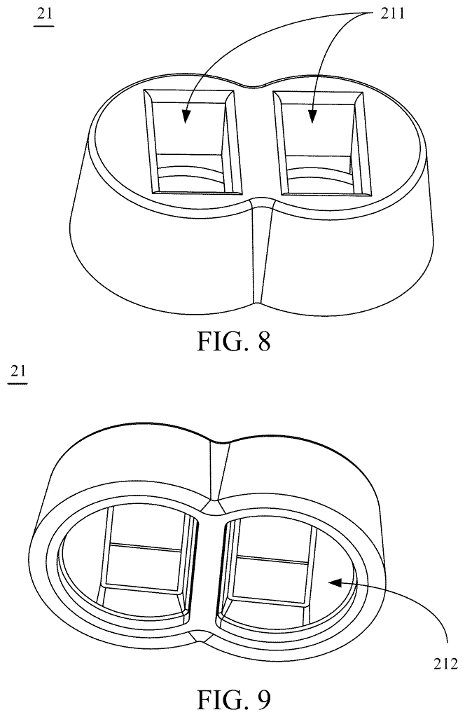

As shown in FIG. 6 and FIG. 7, FIG. 6 is a cross-sectional view of an atomization structure in an A-A direction according to an embodiment of this application; and FIG. 7 is an enlarged view at P in FIG. 6.

In an embodiment, this application provides an atomization structure 2. The atomization structure 2 includes a mounting base 22, at least two microfluidic bodies 23, and a sealing member 21.

The mounting base 22 includes a mounting cavity 221, and the mounting cavity 221 includes a liquid inlet 2211 and a mist outlet 2212. The mounting cavity 221 is configured to accommodate the at least two microfluidic bodies 23 and the sealing member 21. The atomization material enters the mounting cavity 221 from the liquid inlet 2211, and then is sprayed out from the mist outlet 2212 after being atomized by using the microfluidic bodies 23.

The atomization structure 2 includes the at least two microfluidic bodies 23, and the number of the at least two microfluidic bodies may be two, three, or the like. The microfluidic body 23 includes a liquid inlet end 231 and a mist outlet end 232 that are in communication with each other. The liquid inlet end 231 of the microfluidic body 23 is in communication with the liquid inlet 2211 of the mounting cavity 221, and the mist outlet end 232 of the microfluidic body 23 is in communication with the mist outlet 2212 of the mounting cavity 221, so that an atomization material entering from the liquid inlet 2211 is atomized by the at least two microfluidic bodies 23 and then sprayed out from the mist outlet 2212 for the user to inhale. The microfluidic body 23 is a structural member with a plurality of microfluidic channels inside. A liquid atomization material flows from the liquid inlet end 231 to the mist outlet end 232 under an action of pressure, and then may be atomized to form an aerosol after flowing through the inside of the microfluidic body 23. The microfluidic body 23 may generally be made of at least one of a polymer material, a metal, a ceramic, glass, or a silicon-based material.

For ease of understanding and description, the following embodiments of this application are all described by using an example in which the atomization structure 2 includes two microfluidic bodies 23.

The sealing member 21 is arranged in the mounting cavity 221 and seals the at least two microfluidic bodies 23, to prevent liquid leakage during atomization of the atomization material entering the mounting cavity 221 from the liquid inlet 2211 through the microfluidic bodies 23. The sealing member 21 may be made of a flexible material such as silicone.

In this way, the at least two microfluidic bodies 23 are arranged in the atomization structure 2. The liquid atomization material enters the mounting cavity 221 of the mounting base 22 through the liquid inlet 2211 under the action of pressure, enters a microfluidic body through the liquid inlet end 231 of the microfluidic body 23, and is atomized inside the microfluidic body 23 to form an aerosol. The aerosol is sprayed out from the mist outlet 2212 through the mist outlet end 232 for the user to inhale. The at least two microfluidic bodies 23 increase throughput of the atomization material in the mounting cavity 221, and in a case that the pressure is the same, larger throughput of the atomization material indicates a larger amount of atomization.

In an embodiment, as shown in FIG. 8, FIG. 8 is an overall schematic diagram of a sealing member from a first perspective according to an embodiment of this application. The sealing member 21 includes at least two receiving holes 211, and the at least two microfluidic bodies 23 are arranged in the at least two receiving holes 211 in a one-to-one correspondence. In the embodiments of this application, the sealing member 21 includes two receiving holes 211, and each receiving hole 211 runs through two surfaces of the sealing member 21 in the axial direction, in other words, the receiving hole 211 is a through hole. The two receiving holes 211 are spaced apart, and the two microfluidic bodies 23 are arranged in the two receiving holes 211 of the sealing member 21 in a one-to-one correspondence. A shape of the receiving hole 211 is the same as a shape of the microfluidic body 23, so that the sealing member 21 surrounds the two microfluidic bodies 23. A circumferential length of the receiving hole 211 is slightly smaller than a circumferential length of the microfluidic body 23, so that the receiving hole 211 of the sealing member 21 circumferentially squeezes the microfluidic body 23, in other words, the receiving hole 211 is in an interference fit with the microfluidic body 23, to prevent the microfluidic body 23 from falling off from the sealing member 21. In this way, the sealing member 21 can independently seal each microfluidic body 23, and a sealing effect is better.

In an embodiment, the sealing member 21 includes two sub-sealing members in a shape of a cone arranged in parallel and connected to each other on side surfaces, and each sub-sealing member includes an receiving hole 211 in a shape of a cuboid. The sub-sealing member may be coaxially arranged with the receiving hole 211, so that sealing pressure of the sub-sealing member in a shape of a cone is more uniform. The sealing member 21 may be integrally formed, and side surfaces of the two sub-sealing members connected to each other may partially overlap with each other.

In an embodiment, the mounting cavity 221 of the mounting base 22 includes a top wall and a bottom wall, the top wall of the mounting cavity 221 includes the mist outlet 2212, the bottom wall of the mounting cavity 221 includes the liquid inlet 2211, both the sealing member 21 and the two microfluidic bodies 23 are arranged in the mounting cavity 221, the microfluidic bodies 23 are arranged in the receiving holes 211, the sealing member 21 and the bottom wall of the mounting cavity 221 are spaced apart and cooperate to form a shunting cavity 2213, the two receiving holes 211 are respectively located on two opposite sides of an extension line of the liquid inlet 2211, and the two receiving holes 211 are separately in communication with the liquid inlet 2211 through the shunting cavity 2213. In this way, the atomization material enters the shunting cavity 2213 from the liquid inlet 2211, is shunted to the two receiving holes 211 through the shunting cavity 2213, enters the microfluidic bodies 23 from the liquid inlet ends 231, and is atomized, so that a shunting effect is better.

Further, as shown in FIG. 7, the sealing member 21 and the bottom wall of the mounting cavity 221 are spaced apart and cooperate to form the shunting cavity 2213. When the sealing member 21 is deformed inward and tightly attached to the microfluidic body 23 due to a squeeze by the inner wall of the mounting base 22 during assembly, the shunting cavity 2213 is space reserved for deformation of the sealing member 21 in the axial direction, to prevent the sealing member 21 from causing another problem, for example, blocking the liquid inlet end 231, caused by protruding from the liquid inlet end 231 while being deformed inward. In this way, both an atomization effect and a sealing effect are ensured.

The microfluidic body 23 is arranged in the receiving hole 211 of the sealing member 21, and the end surface of the liquid inlet end 231 of the microfluidic body 23 is spaced apart from the bottom wall of the mounting cavity 221. In this way, there is sufficient space to accommodate the atomization material entering from the liquid inlet 2211, and the shunting cavity 2213 is formed to facilitate shunting of the atomization material while avoiding a risk of damage to the microfluidic body 23 caused by the squeeze by the mounting base 22 on the microfluidic body 23 in the circumferential direction.

In an embodiment, structures and sizes of the two microfluidic bodies 23 are the same, both the sealing member 21 and the two microfluidic bodies 23 are arranged in the mounting cavity 221, and the microfluidic bodies 23 are arranged in the receiving holes 211. The sealing member 21 and the bottom wall of the mounting cavity 221 are spaced apart and cooperate to form a shunting cavity 2213. The shunting cavity 2213 includes two same sub-shunting cavities 2213a, and the two microfluidic bodies 23 and the two sub-shunting cavities 2213a are arranged in a one-to-one correspondence. Distances between the two receiving holes 211 and the liquid inlet 2211 are equal. In this way, the atomization material enters the shunting cavity 2213 from the liquid inlet 2211, is evenly shunted to the two sub-shunting cavities 2213a through the shunting cavity 2213, and enters the microfluidic bodies 23 from the liquid inlet ends 231 and is atomized, so that the atomization material is evenly shunted and atomized more evenly.

In an embodiment, as shown in FIG. 9, with reference to FIG. 7, FIG. 9 is an overall schematic diagram of a sealing member from a second perspective according to an embodiment of this application. The surface of the sealing member 21 close to the bottom wall of the mounting cavity 221 includes a first shunting groove 212 or a first protrusion (not shown in the figure), and/or the surface of the bottom wall of the mounting cavity 221 close to the sealing member 21 includes a second shunting groove or a second protrusion. The sealing member 21 is arranged in the mounting cavity 221, and the surface of the sealing member 21 close to the bottom wall of the mounting cavity 221 includes the first shunting groove 212 or the first protrusion. In this way, the bottom wall of the sealing member 21 and the bottom wall of the mounting base 22 cooperate to form the shunting cavity 2213, to shunt the atomization material entering from the liquid inlet 2211; and/or the surface of the bottom wall of the mounting cavity 221 close to the sealing member 21 includes the second shunting groove (not shown in the figure) or the second protrusion (not shown in the figure). Similarly, the bottom wall of the sealing member 21 and the bottom wall of the mounting base 22 cooperate to form the shunting cavity 2213, to shunt the atomization material entering from the liquid inlet 2211, and a shunting effect is better. In this application, an example in which the surface of the sealing member 21 close to the bottom wall of the mounting cavity 221 includes the first shunting groove 212 is used for description, and the two receiving holes 211 are located on the bottom wall of the first shunting groove 212.

In an embodiment, as shown in FIG. 6 and FIG. 7, the number of the mist outlets 2212 of the mounting cavity 221 of the mounting base 22 is at least two, and may be two, three, or the like. The at least two mist outlets 2212 and the mist outlet ends 232 of the at least two microfluidic bodies 23 are arranged in a one-to-one correspondence. It may be understood that the number of the microfluidic bodies 23 is the same as the number of the mist outlets 2212. The number of the liquid inlet 2211 is one. In the embodiments of this application, the number of the mist outlets 2212 of the mounting cavity 221 of the mounting base 22 is two, both the sealing member 21 and the two microfluidic bodies 23 are arranged in the mounting cavity 221, the microfluidic bodies 23 are arranged in the receiving holes 211, and the two mist outlets 2212 and the mist outlet ends 232 of the two microfluidic bodies 23 are arranged in a one-to-one correspondence. In this way, the aerosol formed after the atomization material is atomized by the microfluidic body 23 is sprayed out from the mist outlet end and then directly sprayed out through the mist outlet 2212 corresponding to the microfluidic body 23, and there is no blocking on a mist outlet path, thereby improving atomization efficiency. The number of the liquid inlet 2211 of the mounting cavity 221 is one, which facilitates production of the mounting cavity 221. In addition, the number of the liquid inlet 2211 is reduced as much as possible, thereby improving sealing performance of the mounting cavity 221.

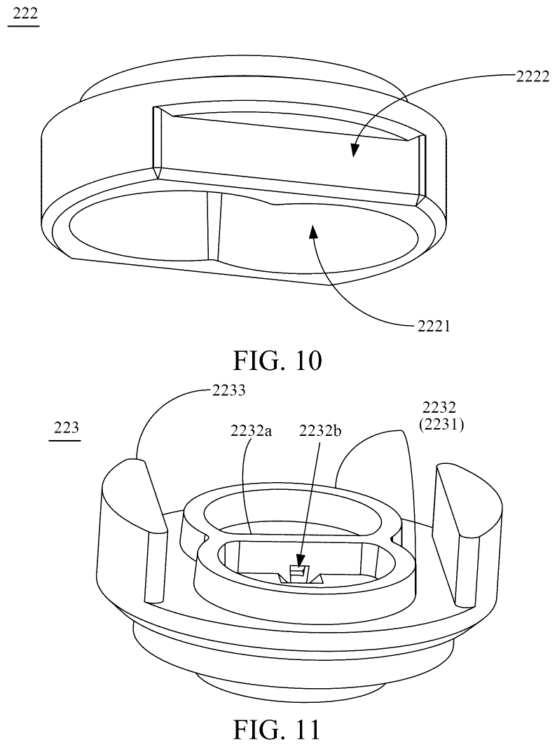

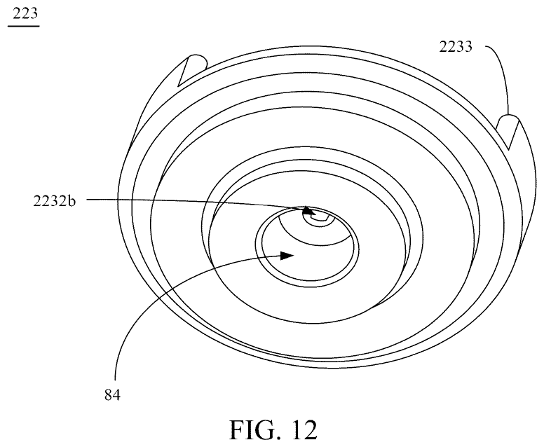

As shown in FIG. 10 and FIG. 11, FIG. 10 is an overall schematic diagram of a first mounting base according to an embodiment of this application, and FIG. 11 is an overall schematic diagram of a second mounting base from a first perspective according to an embodiment of this application. In an embodiment, with reference to FIG. 6 and FIG. 7, the mounting base 22 includes a first mounting base 222 and a second mounting base 223. The first mounting base 222 includes a mounting groove 2221; and the second mounting base 223 covers the mounting groove 2221 to form the mounting cavity 221. A flange 2231 is arranged on the surface of the second mounting base 223 close to the first mounting base 222. The flange 2231 is inserted into the mounting groove 2221. The sealing member 21 abuts against the flange 2231 and is spaced apart from the surface of the second mounting base 223 close to the first mounting base 222. The sealing member 21 is squeezed into the mounting cavity 221 by the flange 2231, and the sealing member 21 is squeezed from the side of the liquid inlet end 231 of the microfluidic body 23, so that the sealing member 21 is deformed inward, thereby sealing the microfluidic bodies 23 surrounded by the sealing member 21, and preventing liquid leakage when the atomization material flows to the microfluidic bodies 23. It may be understood that the sealing member 21 can be deformed by the flange 2231, and the deformation can convert axial pressure pointing to the sealing member 21 from the flange 223 1 into pressure surrounding the microfluidic body 23, in other words, convert the axial pressure into pressure in a planar direction perpendicular to the microfluidic body 23, which improves stability of fixing of the microfluidic body 23 and a sealing effect. In addition, the pressure is evenly dispersed, and damage to the microfluidic body 23 due to concentrated pressure is avoided.

In an embodiment, the flange 2231 defines two adjacent convex rings 2232, and each convex ring 2232 is arranged corresponding to one of the receiving holes 211. The end portion of each convex ring 2232 abuts against the end surface of the side wall of a corresponding receiving hole 211, so that the two sub-shunting cavities 2213a are independent of each other, and an amount of the aerosol formed through atomization by the two microfluidic bodies 23 is relatively constant. A common side wall 2232a of the two convex rings 2232 includes an opening 2232b for shunting liquid from the liquid guide tube 4.

Further, the cross section of the flange 2231 is trapezoidal or square, and the flange 2231 has no sharp corners, so that a squeezing force is uniformly applied to the sealing member 21, and the sealing member 21 can be uniformly deformed and sealed and fixed.

In an embodiment, as shown in FIG. 10 and FIG. 11, the outer side surface of the first mounting base 222 includes a limiting groove 2222. The flange 2231 is an annular flange 2231. The surface of the second mounting base 223 close to the first mounting base 222 includes a limiting side wall 2233 located on the outer side of the annular flange 2231. The limiting side wall 2233 is spaced apart from the annular flange 2231, and the limiting side wall 2233 is inserted into the limiting groove 2222. In this way, through limiting and clamping between the limiting groove 2222 and the limiting side wall 2233, the first mounting base 222 and the second mounting base 223 cooperate to form the mounting cavity 221, thereby preventing the first mounting base 222 and the second mounting base 223 from being displaced.

In an embodiment, as shown in FIG. 2, FIG. 6, and FIG. 7, the atomization structure 2 further includes a holder 8 and a filter element 85. The holder 8 includes a receiving cavity 81, the mounting base 22 is arranged in the receiving cavity 81, and the receiving cavity 81 includes a liquid inlet channel 82 in communication with the liquid inlet 2211, and an avoidance opening 83 for exposing the mist outlet 2212. The filter element 85 is arranged at a joint between the liquid inlet channel 82 and the liquid inlet 2211, to filter the atomization material flowing to the microfluidic body 23, and the liquid inlet channel 82 is in communication with the liquid inlet 2211 through the filter element 85. A groove 84 may be provided on the mounting base 22 for arranging the filter element 85, or a groove 84 may be provided on the holder 8 for arranging the filter element 85. The liquid inlet channel 82 is in communication with the liquid guide tube 4. In this way, the atomization material in the liquid storage bottle 7 is guided by the liquid guide tube 4 to the liquid inlet channel 82 under the action of pressure, then enters the liquid inlet 2211 through the filter element 85, and is atomized by the microfluidic body 23 to form an aerosol. The aerosol passes through the mist outlet 2212 and the avoidance opening 83 and is finally inhaled by the user.

In an embodiment, as shown in FIG. 11 and FIG. 12, FIG. 12 is an overall schematic diagram of a second mounting base from a second perspective according to an embodiment of this application. A groove 84 is provided on the surface of the second mounting base 223 away from the first mounting base 222 for arranging the filter element 85, and the bottom of the groove 84 is in communication with an opening 2232b of the common side wall 2232a of the two convex rings 2232 correspondingly.

In some embodiments, the atomization structure 2 further includes a buffer gasket (not shown in the figure), and the buffer gasket is arranged between the sealing member 21 and the first mounting base 222. In this way, the sealing member 21 is lifted to prevent the microfluidic body 23 surrounded by the sealing member 21 from being in rigid contact with the first mounting base 222, and to prevent the microfluidic body 23 from moving upward and being damaged due to a rigid squeeze generated between the microfluidic body and the first mounting base 222 when the second mounting base 223 is mounted.

In an embodiment, as shown in FIG. 4a, the housing includes a mist outlet channel 31, one end of the atomization structure 2 is located in the mist outlet channel 31 and includes the mist outlet 2212, the mist outlet 2212 is spaced apart from a port 311 of the mist outlet channel 31, and the cover body 5 is rotatably connected to the housing 3. When the cover body 5 is configured to be in the first state, the cover body 5 covers the mist outlet channel 31 and is engaged with the housing 3, and the blocking member 6 is located between the cover body 5 and the mist outlet 2212. In this way, the blocking member 6 is squeezed between the cover body 5 and the mist outlet 2212, and the mist outlet 2212 is blocked more tightly. When the cover body 5 is configured to be in the second state, the mist outlet channel 31 is exposed.

This application provides an inhalation device 1, and the device includes: a housing 3; an atomization structure 2, arranged in the housing 3 and including a mist outlet 2212; a cover body 5, movably connected to the housing 3; and a blocking member 6, arranged on the cover body 5, where when the cover body 5 is configured to be in a first state, the blocking member 6 is attached to the mist outlet 2212 and blocks the mist outlet 2212; and when the cover body 5 is configured to be in a second state, the blocking member 6 is separated from the mist outlet 2212. In this way, when the cover body 5 is configured to be in the first state, blocking member 6 is attached to the mist outlet 2212 and blocks the mist outlet 2212, so that the atomization material in the housing 3 does not evaporate through the mist outlet 2212 after passing through the atomization structure 2. In addition, the mist outlet 2212 can be prevented from being polluted, and a problem of a large deviation in the amount of mist sprayed out can be reduced, to maintain consistency in an amount of atomization as much as possible. In addition, when the inhalation device 1 is used again after being unused for a period of time, the deviation in the amount of mist sprayed out is small, and there are fewer pre-sprays, thereby improving user experience.

The descriptions are merely implementations of this application, and the patent scope of this application is not limited thereto. All equivalent structure or process changes made according to the content of this specification and the accompanying drawings in this application or by directly or indirectly applying this application in other related technical fields shall fall within the protection scope of this application.

1. An inhalation device, comprising:
a housing;
an atomization structure arranged in the housing and comprising a mist outlet;
a cover body movably connected to the housing; and
a blocking member arranged on the cover body,
wherein when the cover body is configured to be in a first state, the blocking member is attached to the mist outlet and blocks the mist outlet; and when the cover body is configured to be in a second state, the blocking member is separated from the mist outlet.

2. The inhalation device of claim 1, wherein the blocking member is detachably connected to the cover body.

3. The inhalation device of claim 2, wherein a first receiving groove is provided on the inner wall surface of the cover body, and the blocking member is embedded in the first receiving groove; or
a second receiving groove is provided on the blocking member, a convex clamping block is arranged on the inner wall surface of the cover body, and the clamping block is embedded in the second receiving groove.

4. The inhalation device of claim 2, wherein a first magnetic member is arranged on the cover body, a second magnetic member is arranged on the blocking member, and the blocking member is magnetically connected to the cover body.

5. The inhalation device of claim 1, wherein the surface of the blocking member comprises a bump, wherein when the cover body is configured to be in the first state, the bump is embedded in the mist outlet and is in an interference fit with the mist outlet.

6. The inhalation device of claim 1, wherein the atomization structure comprises:
a mounting base comprising a mounting cavity, wherein the mounting cavity comprises a liquid inlet and the mist outlet;
at least two microfluidic bodies arranged in the mounting cavity; and
a sealing member arranged in the mounting cavity and sealing the at least two microfluidic bodies,
wherein a liquid inlet end of the microfluidic body is in communication with the liquid inlet, and a mist outlet end of the microfluidic body is in communication with the mist outlet to cause an aerosol-generating material entering from the liquid inlet to be sprayed out from the mist outlet after being atomized by the at least two microfluidic bodies.

7. The inhalation device of claim 6, wherein the sealing member comprises at least two receiving holes, and the at least two microfluidic bodies are arranged in the at least two receiving holes in a one-to-one correspondence;
the mounting cavity comprises a top wall and a bottom wall, wherein the top wall comprises the mist outlet, and the bottom wall comprises the liquid inlet; and
the sealing member and the bottom wall of the mounting cavity are spaced apart and cooperate to form a shunting cavity, and the two receiving holes are respectively located on two opposite sides of an extension line of the liquid inlet and are separately in communication with the liquid inlet through the shunting cavity.

8. The inhalation device of claim 7, wherein the mounting base comprises:
a first mounting base comprising a mounting groove; and
a second mounting base covering the mounting groove to form the mounting cavity,
wherein a flange is arranged on the surface of the second mounting base close to the first mounting base, the flange is inserted into the mounting groove, and the sealing member abuts against the flange and is spaced apart from the surface of the second mounting base proximate to the first mounting base.

9. The inhalation device of claim 6, wherein the atomization structure further comprises:
a holder comprising a receiving cavity, wherein the mounting base is arranged in the receiving cavity; and the receiving cavity comprises
a liquid inlet channel in communication with the liquid inlet, and
an avoidance opening for exposing the mist outlet; and
a filter element arranged at a joint between the liquid inlet channel and the liquid inlet, wherein the liquid inlet channel is in communication with the liquid inlet through the filter element.

10. The inhalation device of claim 1, wherein the housing comprises a mist outlet channel, one end of the atomization structure is located in the mist outlet channel and comprises the mist outlet, and the mist outlet is spaced apart from a port of the mist outlet channel, and the cover body is rotatably connected to the housing,
wherein when the cover body is configured to be in the first state, the cover body covers the mist outlet channel and is engaged with the housing; and when the cover body is configured to be in the second state, the mist outlet channel is exposed.
